(19) United States
(12) Patent Application Publication
(10) Pub. No.: US 2020/0190469 A1
(43) Pub. Date: Jun. 18, 2020

(54) Venus Protocol Method for Enhancing Female Sexual Wellness with Shockwave Therapy and Stem Cells

(71) Applicant: Joy Kong, Chatsworth, CA (US)

(72) Inventor: Joy Kong, Chatsworth, CA (US)

(21) Appl. No.: 16/218,192

(22) Filed: Dec. 12, 2018

(51) Int. Cl. C12N 5/074; A61P 15/12; A61P 15/02; A61H 23/00; A61K 31/167; A61K 9/00; C12N 5/078; A61M 5/30

(52) U.S. Cl. CPC C12N 5/0607; A61P 15/12; A61P 15/02; A61M 5/30; A61K 31/167; A61K 9/0019; C12N 5/0644; A61H 23/008

(57) ABSTRACT

A method for enhancing female sexual wellness with shockwave therapy and stem cells promotes tissue repair and regeneration in or near the female vagina through the combinative effects of shockwave therapy and needle-free injection of a stem cell specimen on the genital. The method includes: preparing the patient, both mentally and physically to receive stem cell therapy; applying shockwave therapy to the genitalium; developing a platelet-rich plasma preparation from the patient; introducing a stem cell specimen into a vial having a needle-free injector; introducing the platelet-rich plasma preparation into the vial; injecting, through the needle-free injector, the stem cell specimen and the platelet-rich plasma preparation directly on or near the vagina and through the vaginal canal from multiple positions, multiple depths, and multiple angles; reapplying shockwave therapy to the vagina after a duration; and monitoring the female patient for a post-procedure duration after administration of the stem cell specimen.

### FIELD OF THE INVENTION

The present invention relates generally to a Venus protocol method for enhancing female sexual wellness with shockwave therapy and stem cells. More so, the present invention relates to a method for enhancing female sexual wellness with shockwave therapy and stem cells; whereby the method promotes tissue repair and regeneration in or near the genitalia, i.e., female vagina, through the combinative effects of shockwave therapy and needle-free injection of a stem cell specimen directly on the vagina; and whereby needle-free administration of stem cells allows for a low anxiety, painless entry, and a more uniform distribution of the deposited stem cell specimen.

### BACKGROUND OF THE INVENTION

The following background information may present examples of specific aspects of the prior art (e.g., without limitation, approaches, facts, or common wisdom) that, while expected to be helpful to further educate the reader as to additional aspects of the prior art, is not to be construed as limiting the present invention, or any embodiments thereof, to anything stated or implied therein or inferred thereupon.

It is known that the genitalia, or sex organ, is a part of the body that is involved in sexual reproduction. The reproductive organs together constitute the reproductive system. The ovary in the female is one of the primary sex organs. The vagina is a muscular canal lined with nerves and mucus membranes. It connects the uterus and cervix to the outside of the body, allowing for menstruation, intercourse, and childbirth. The vaginal opening, also called the vaginal vestibule or introitus, is the opening into the vagina. The vaginal opening is located between the urethra and the anus. The vaginal opening is where menstrual blood leaves the body. The vaginal wall is made of muscle covered in a mucus membrane, similar to the tissue in your mouth. The wall contains layers of tissue with many elastic fibers.

Typically, stem cells are primitive cells in our body serving as a reservoir, able to replenish itself and differentiate into a wide range of specialized cells, in order to replace damaged cells and regenerate tissue. In addition, stem cells can be collected and prepared from a wide range of sources that can include, but are not limited to, autologous sources such as a person's own body, including fat, bone marrow or blood, or even umbilical cord and other birth tissues. Stem cells have innate intelligence, able to home in to injured areas, secrete bioactive molecules that exert local and systemic effects, reduce inflammation, and recruit local cells to assist in the healing process. One common stem cell specimen is a mesenchymal stem cell.

Other proposals have involved methods for treating the vagina with stem cell specimens. The problem with these methods is that they do not incorporate shockwave therapy with the stem cells. Also, the prior art methods do not inject mesenchymal stem cells directly into the vagina wall. Even though the above cited methods for administering stem cell specimens meet some of the needs of the market, a method for enhancing female sexual wellness with shockwave therapy and stem cells to promote tissue repair and regeneration in or on the outer surface of the female vagina through the combinative effects of shockwave therapy and needle-free injection of a stem cell specimen directly on and inside the vagina, is still desired.

### SUMMARY

Illustrative embodiments of the disclosure are generally directed to a Venus protocol method for enhancing female sexual wellness with shockwave therapy and stem cells. The method promotes tissue repair and regeneration in or near the genitalia, i.e., female vagina through the combinative effects of shockwave therapy and needle-free injection of a stem cell specimen directly on, and in the vaginal cavity of the vagina. The needle-free administration of stem cells allows for a low anxiety, painless entry, and a more uniform distribution of the deposited stem cell specimen. The method may be helpful in conditions such as inorgasmia, vaginal dryness, dyspareunia, urinary incontinence, and may improve female sexual arousal and quality of orgasms.

In one embodiment, the method involves a series of steps, including: preparing the patient, both mentally and physically to receive stem cell therapy; applying shockwave therapy to the vagina; developing a platelet-rich plasma preparation from the patient; introducing a stem cell specimen into a vial having a needle-free injector; introducing the platelet-rich plasma preparation into the vial.

The method also includes: injecting, through the needle-free injector, the stem cell specimen and the platelet-rich plasma preparation directly on or near the vagina from multiple positions, multiple depths, and multiple angles; reapplying shockwave therapy to the vagina after a duration; and monitoring the patient for a post-procedure duration after administration of the stem cell specimen and the platelet-rich plasma preparation.

The method for enhancing female sexual wellness with shockwave therapy and stem cell therapy includes an initial Step of obtaining consent from the patient to proceed with the method.

The method may include an initial Step of obtaining at least one metric of a patient.

The method may further comprise a Step of applying shockwave therapy to the vagina of the patient.

A Step includes developing a platelet-rich plasma preparation from the patient.

In some embodiments, a Step comprises obtaining a stem cell specimen.

A Step includes introducing the stem cell specimen into a vial, the vial having a detachably attached needle-free injector defined by multiple nozzles.

In some embodiments, a Step may include introducing the platelet-rich plasma preparation into the vial.

A Step comprises drawing about 0.5 cubic centimeters of the stem cell specimen and the platelet-rich plasma preparation from the vial into each nozzle of the needle-free injector.

The method may further comprise a Step of injecting, through at least one of the nozzles of the needle-free injector, the stem cell specimen and the platelet-rich plasma preparation directly on or near the vagina from multiple positions, multiple depths, and multiple angles, whereby multiple injections are performed.

A Step includes reapplying, after a duration, the shockwave therapy to the vagina.

A final Step includes monitoring the patient for a post-procedure duration after administration of the stem cell specimen and the platelet-rich plasma preparation.

In another aspect, the method further comprises a step of activating the platelet-rich plasma by positioning the platelet-rich plasma in a 37° Fahrenheit water bath for 1 to 3 minutes.

In another aspect, the method further comprises a step of applying an ultrasound gel over the vagina.

In another aspect, the method further comprises a step of applying a numbing cream over the vagina.

In another aspect, the method further comprises a step of heating a stem cell specimen vial containing the stem cell specimen to induce thawing by emerging the stem cell specimen vial in a 37° water bath, or by rolling the stem cell specimen vial against the palms of the hands.

In another aspect, the step of introducing the stem cell specimen into a vial, further comprises drawing 2 cubic centimeters of the stem cell specimen into a 5 cubic centimeter syringe, the syringe being detachably attached to a 18 G 1½″ needle.

In another aspect, the step of introducing the platelet-rich plasma preparation into the vial, further comprises drawing 1 cubic centimeter of the platelet-rich plasma preparation into the 5 cubic centimeter syringe, whereby the stem cell specimen and the platelet-rich plasma preparation are mixed.

In another aspect, the method further comprises a step of puncturing a rubber top on the vial with the needle-free injector.

In another aspect, the needle-free injector comprises six nozzles.

In another aspect, the genitalium comprises a vagina.

In another aspect, the method further comprises a step of cleaning the vulval area of the vagina with a povodone-iodine swabstick.

In another aspect, the method further comprises a step of spreading open the vaginal canal of the vagina with a speculum.

In another aspect, the step of injecting, through the needle-free injector, the stem cell specimen and the platelet-rich plasma preparation directly on or near the vagina from multiple positions, multiple depths, and multiple angles, further comprises injecting, through at least one of the nozzles of the needle-free injector, the stem cell specimen and the platelet-rich plasma preparation at the open vaginal canal at a 90° angle relative to the urethral plane of the vagina.

In another aspect, the step of injecting, through the needle-free injector, the stem cell specimen and the platelet-rich plasma preparation directly on or near the vagina from multiple positions, multiple depths, and multiple angles, further comprises injecting, through at least one of the nozzles of the needle-free injector, the stem cell specimen and the platelet-rich plasma preparation at the open vaginal canal at a 90° angle relative to the urethral plane of the vagina.

One objective of the present invention is to use stem cells to help repair damaged tissues and repair injuries near the vagina.

Another objective is to use a shockwave device to generate shock waves which can be adjusted for intensity, frequency and modulation so as to maximize therapeutic effects.

Another objective is to reduce local inflammation, improve blood flow, promote new blood vessel formation, facilitate breaking down of scar tissue, stimulate growth factor release, and activate local stem cell population.

Another objective is to promoting tissue regeneration in and around the vagina.

Another objective is to prevent infections in the vagina through use of a Povidone-iodine swab sticks.

Another objective is to ensure the mental well-being of the female patient prior to the stem cell therapy.

Other systems, devices, methods, features, and advantages will be or become apparent to one with skill in the art upon examination of the following drawings and detailed description. It is intended that all such additional systems, methods, features, and advantages be included within this description, be within the scope of the present disclosure, and be protected by the accompanying claims and drawings.

### BRIEF DESCRIPTION OF THE DRAWINGS

The invention will now be described, by way of example, with reference to the accompanying drawings, in which:

FIG. 1 illustrates a flowchart of an exemplary Venus protocol method for enhancing female sexual wellness with shockwave therapy and stem cells, in accordance with an embodiment of the present invention;

FIG. 2 illustrates a sectioned side view of the female abdomen, showing the vagina, in accordance with an embodiment of the present invention;

FIG. 3 illustrates a perspective view of fingers spreading the vagina canal, in accordance with an embodiment of the present invention;

FIG. 4 illustrates a top view of the female vagina, showing the angles and positions of the needle-free injector for injecting stem cell specimen, in accordance with an embodiment of the present invention;

FIG. 5 illustrates a sectioned side view of the female abdomen, showing a shockwave device, in accordance with an embodiment of the present invention; and

FIG. 6 illustrates an anatomical depiction of a full frontal view of the female vagina from the inner thigh to the anus, in accordance with an embodiment of the present invention.

Like reference numerals refer to like parts throughout the various views of the drawings.

### DETAILED DESCRIPTION OF THE INVENTION

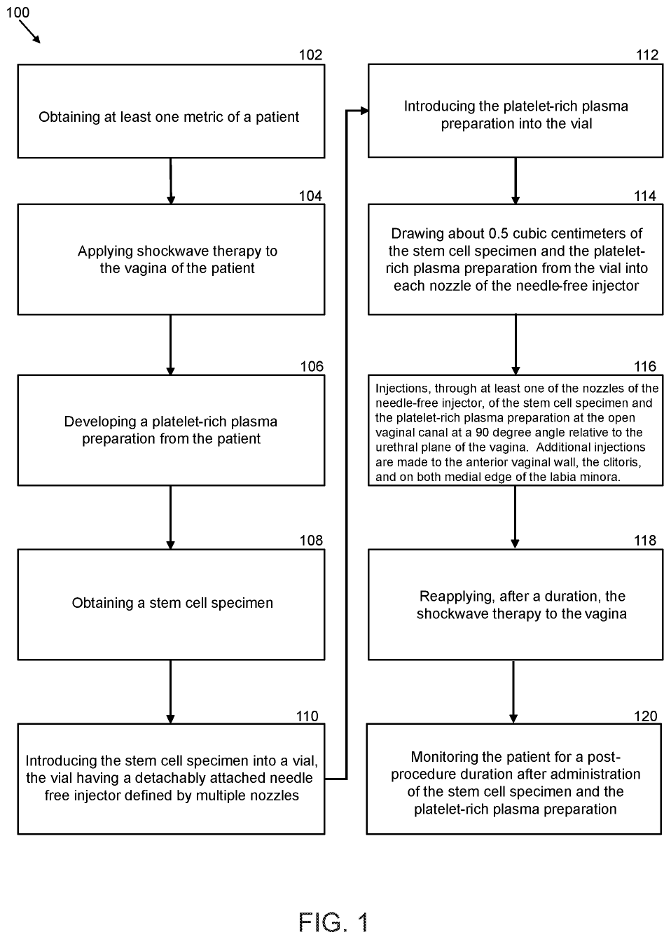

The following detailed description is merely exemplary in nature and is not intended to limit the described embodiments or the application and uses of the described embodiments. As used herein, the word “exemplary” or “illustrative” means “serving as an example, instance, or illustration.” Any implementation described herein as “exemplary” or “illustrative” is not necessarily to be construed as preferred or advantageous over other implementations. All of the implementations described below are exemplary implementations provided to enable persons skilled in the art to make or use the embodiments of the disclosure and are not intended to limit the scope of the disclosure, which is defined by the claims. For purposes of description herein, the terms “upper,” “lower,” “left,” “rear,” “right,” “front,” “vertical,” “horizontal,” and derivatives thereof shall relate to the invention as oriented in FIG. 1. Furthermore, there is no intention to be bound by any expressed or implied theory presented in the preceding technical field, background, brief summary or the following detailed description. It is also to be understood that the specific devices and processes illustrated in the attached drawings, and described in the following specification, are simply exemplary embodiments of the inventive concepts defined in the appended claims. Specific dimensions and other physical characteristics relating to the embodiments disclosed herein are therefore not to be considered as limiting, unless the claims expressly state otherwise.

A Venus protocol method 100 for enhancing female sexual wellness with shockwave therapy and stem cells is referenced in FIGS. 1-6. The method 100 promotes tissue repair and regeneration in or near the genitalia, such as the vagina. The method 100 is operable with the female vagina. The method 100 creates a synergy between shockwave therapy and needle-free injection of a stem cell specimen directly on the vagina. It is unique that the needle-free administration of stem cells allows for a low anxiety, painless entry, and a more uniform distribution of the deposited stem cell specimen. The method 100 is efficacious for healing conditions, such as inorgasmia, vaginal dryness, dyspareunia, urinary incontinence, and may improve sexual arousal and quality of orgasms in the vagina.

The method 100 generally includes a series of steps that both prepare the genitalia/vagina for shockwave therapy and stem cell deposition. The method 100 includes: preparing the patient, both mentally and physically to receive stem cell therapy; applying shockwave therapy to the genitalium; developing a platelet-rich plasma preparation from the patient; introducing a stem cell specimen into a vial having a needle-free injector; and introducing the platelet-rich plasma preparation into the vial.

The method 100 also includes injecting, through the needle-free injector, the stem cell specimen and the platelet-rich plasma preparation directly on or near the vagina from multiple positions, multiple depths, and multiple angles; reapplying shockwave therapy to the genitalium after a duration; and monitoring the patient for a post-procedure duration after administration of the stem cell specimen and the platelet-rich plasma preparation.

The method 100 is effective for reducing local inflammation, improving blood flow, promoting new blood vessel formation, facilitating breaking down of scar tissue, stimulating growth factor release, and activating local stem cell population at the vagina 200. It is known in the art that the vagina is a muscular canal lined with nerves and mucus membranes that connects the uterus 202 and cervix to the outside of the body, allowing for menstruation, intercourse, and childbirth. Thus, the method 100 is also efficacious for promoting tissue regeneration in and around the vagina. Other benefits include using stem cells to help repair damaged tissues and repair injuries near the vagina; and using a shockwave device to generate shock waves which can be adjusted for intensity, frequency and modulation so as to maximize therapeutic effects.

The method 100, as described below, requires a unique needle-free injector with multiple nozzles, stem cell specimens, topical creams, and syringes to properly administer the stem cell specimen and the platelet-rich plasma preparation on the vagina. One requirement can include a CharaCore™ stem cell specimen. In one embodiment, the CharaCore™ can be 2 cc of the actual stem cell specimen having a substantial amount of mesenchymal stem cells.

It is known in the art that CharaCore™ contains 8-10 million cells/cc. These are “native stem cells.” Research shows that native mesenchymal stem cells are at least 10 times more potent than expanded MSC's. Further, Characore™ exhibits a high cell count, with valuable components from umbilical cord blood, cord tissue and amniotic membrane. Delicately processed, thus maximally preserving therapeutic elements. Characore™ contains a high amount of MSC's and various growth factors. Characore™ is offered in 1 cc and 2 cc vials for all forms of injections; with the present invention utilizing the 2 cc vial.

The method 100 also requires an ultrasound gel for the shockwave device, and a BLT (Benzocaine, Lidocaine, Tetracaine) topical anesthetic cream for numbing the treatment area around the vagina. For administrating the stem cell specimen and the platelet-rich plasma preparation, the method 100 utilizes one Lenis needle-free injector (or 2 for fasting loading); one Lenis adapter for drawing liquid into six nozzles; and six Lenis injector nozzles.

The method 100 also contains the stem cell specimen and the platelet-rich plasma preparation in a 10 cc sterile empty vial with rubber stopper, and injects through a 5 cc syringe and an 18 G 1½″ needle. One objective of the method 100 is to prevent infections on the vagina. Thus, the method 100 may also require a Povidone-iodine swabsticks and alcohol swabs to clean the vulva and vagina.

The present method 100 can generate revenue in numerous ways. For example, in order for a healthcare provider to provide treatment for the female sexual wellness treatment (Venus Lift), or the intranasal procedure (Brain Reboot), they will have to pay a licensing fee of: $100 per month as long as they are providing treatment or using those trademarked phrases on their website or other marketing material.

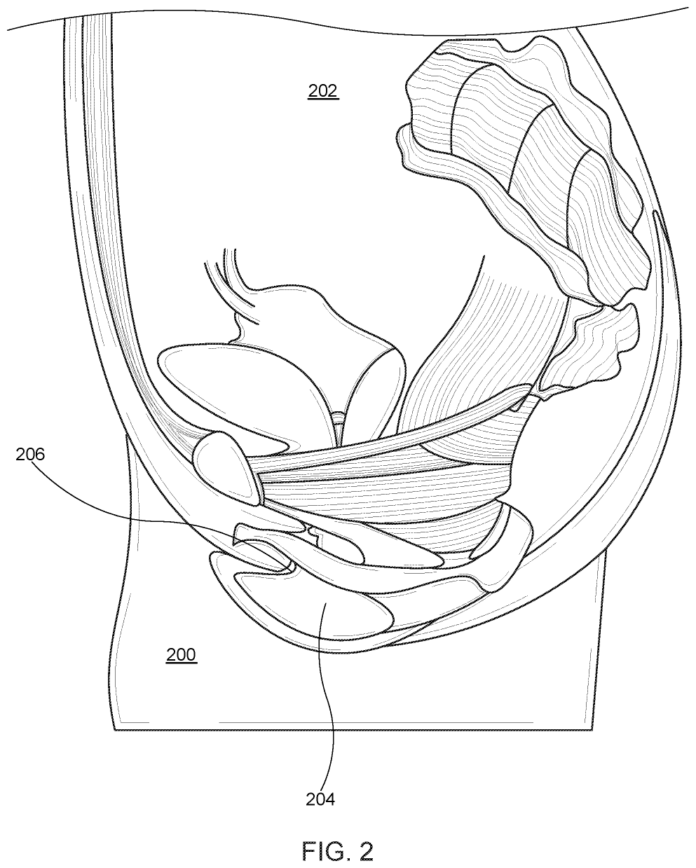

FIG. 1 illustrates a flowchart diagram of an exemplary method 100 for enhancing female sexual wellness with shockwave therapy and stem cells. The method 100 is operable with a female vagina 200 (FIG. 2) to enhance wellness thereto. The method 100 may include an initial Step 102 of obtaining at least one metric of a patient. The metric of a patient may include, without limitation, body temperature, blood pressure, and pulse oximetry measurement. Though in other embodiments, different metrics may be measured.

In some embodiments, the method 100 also includes a step of obtaining consent to proceed with the method 100 from the patient. Standard consent forms commonly used in the medical industry may be used for this purpose. In one embodiment, the method 100 requires a clinical evaluation of the patient to determine appropriateness for shockwave and stem cell therapy to the vagina. The clinic collects a detailed medical history and provides a physical exam of the patient.

In order to prepare the vagina for application of a shockwave device, the method comprises a step of applying an ultrasound gel over the vagina and vulva area. In one embodiment, the ultrasound gel is applied over entire outer vaginal area. The shock wave device is positioned over the clitoral region and the entire vulval area of the vagina. A medium intensity is applied with intermittent high intensity as tolerated by the patient, for 15 minutes using a slow circular motion on the vagina.

Further, a numbing cream is applied over the genitalium to reduce pain and swelling during stem cell deposition. This requires cleaning off the ultrasound gel, and applying a BLT numbing cream in treatment area, which can be over clitoris, the vicinity of urethra, and anterior and lateral vaginal walls 206 up to 2″ into the vaginal canal. The topical anesthetic numbing cream is allowed to remain for about 30 minutes, so as to be absorbed by the vagina sufficiently.

The method 100 may further comprise a Step 104 of applying shockwave therapy to the vagina of the patient. This can be performed with a shockwave device. Those skilled in the art will recognize that shockwave treatment works by using radial acoustic shockwaves to stimulate self-healing. The generated shockwave goes through the tissue and stimulates the cells dealing with connective tissue and bone healing. By stimulating these cells, the body's natural self-healing mechanism is triggered.

In one embodiment, the method has an Initial Phase that requires two sessions for maximal results, about 8-12 weeks apart. In each session, 2 cc CharaCore™ is required. Ideally, shockwave therapy is recommend immediately prior to stem cell injections, for ten minutes at medium strength. This can be applied twenty-four hours after stem cell injections, and then two times per week for six weeks, apply shockwave therapy using in-home device. Each time, a medium intensity shockwave is applied. However, the shockwave can also be alternated with high intensity shockwaves as the patient can tolerate, for about 15 minutes.

A Step 106 includes developing a platelet-rich plasma preparation from the patient. This may involve drawing blood from the patient through intravenous means to prepare for platelet-rich plasma. This process could take approximately 20 minutes. In one non-limiting embodiment, the platelet-rich plasma is activated by placing in a 37° water bath for 1-3 minutes. In one alternative embodiment, the method includes a step of activating the platelet-rich plasma by positioning the platelet-rich plasma in a 37° Fahrenheit water bath for 1 to 3 minutes.

In some embodiments, a Step 108 comprises obtaining a stem cell specimen. In one embodiment, the stem cell specimen is in a stem cell specimen vial, which is stored in dry ice. In another exemplary collection of the stem cell specimen, the stem cell specimen is removed from dry ice, and a peel off product sticker is affixed to a medical record file. The stem cell specimen is prepared for use by heating a stem cell specimen vial containing the stem cell specimen.

This induces thawing by emerging the stem cell specimen vial in a 37° Fahrenheit water bath, or by rolling the stem cell specimen vial against the palms of the hands. Either manner heats the stem cell specimen sufficiently for pouring into a vial, as described below. The stem cell specimen is the content by putting vial in 37° Celsius water bath (or by holding vial in hand, gently rolling against skin). It generally takes 2-2½ minutes for complete thawing via water bath, and 4-5 minutes by hand.

A Step 110 includes introducing the stem cell specimen into a vial, the vial having a detachably attached needle-free injector defined by multiple nozzles 410. In some embodiments, 2 cubic centimeters of the stem cell specimen is drawn into a 5 cubic centimeter syringe, the syringe being detachably attached to an 18 G 1½″ needle. Also, 1 cubic centimeter of the platelet-rich plasma preparation is drawn into the 5 cubic centimeter syringe. In this manner, the stem cell specimen and the platelet-rich plasma preparation are mixed in the vial.

Further, the needle-free injector comprises one Lenis adapter for drawing liquid into six nozzles; and six Lenis injector nozzles. Though other types of nozzles may be used. As described below, each nozzle will administer a dosage of stem cell specimen and platelet-rich plasma preparation to a different section, angle, and depth of the vagina.

In some embodiments, a Step 112 may include introducing the platelet-rich plasma preparation into the vial. It is significant to note that the vial may have a rubber cap; and thereby require a step of puncturing a rubber top on the vial with the needle-free injector. Thus, it is necessary to puncture the rubber top of the vial with the needle-free adapter, and make sure adapter is sitting securely over the vial.

A Step 114 comprises drawing about 0.5 cubic centimeters of the stem cell specimen and the platelet-rich plasma preparation from the vial into each nozzle of the needle-free injector. The needle-free injector is cocked to the side and a pre-filled nozzle is attached into tip of the injector. It is necessary to attach the nozzle securely onto the adapter, and invert the vial. Then, 0.5 cc of stem cell prep is drawn into each of the 6 nozzles.

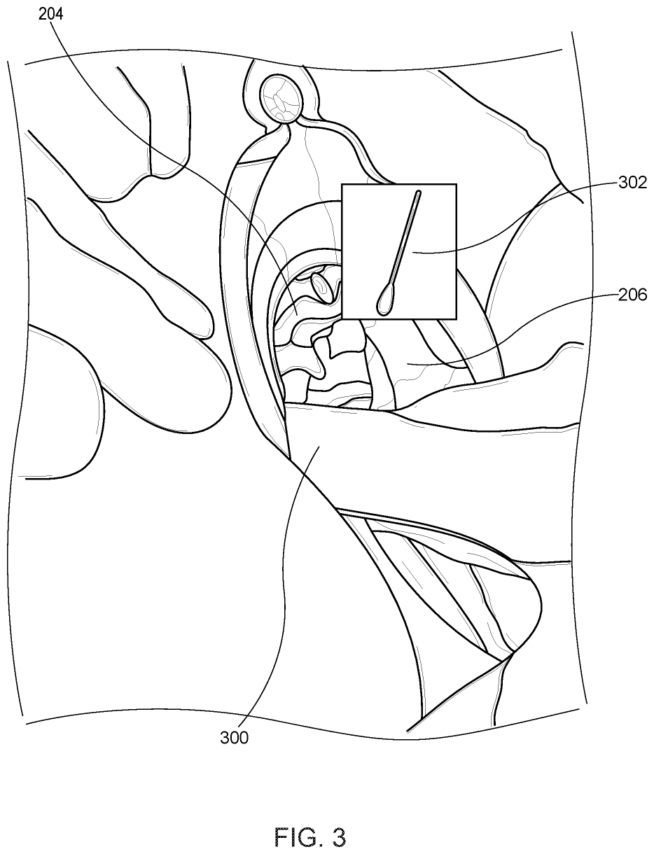

In some embodiments, an additional Step includes cleaning the vulval area and the vaginal opening with a povodone-iodine swabstick 302. A possible Step for the female sexual wellness may include spreading open the vaginal canal 204. Though the fingers 300 of the medical professional may also be used to open the vaginal opening to expose the vaginal canal 204, as shown in FIG. 3.

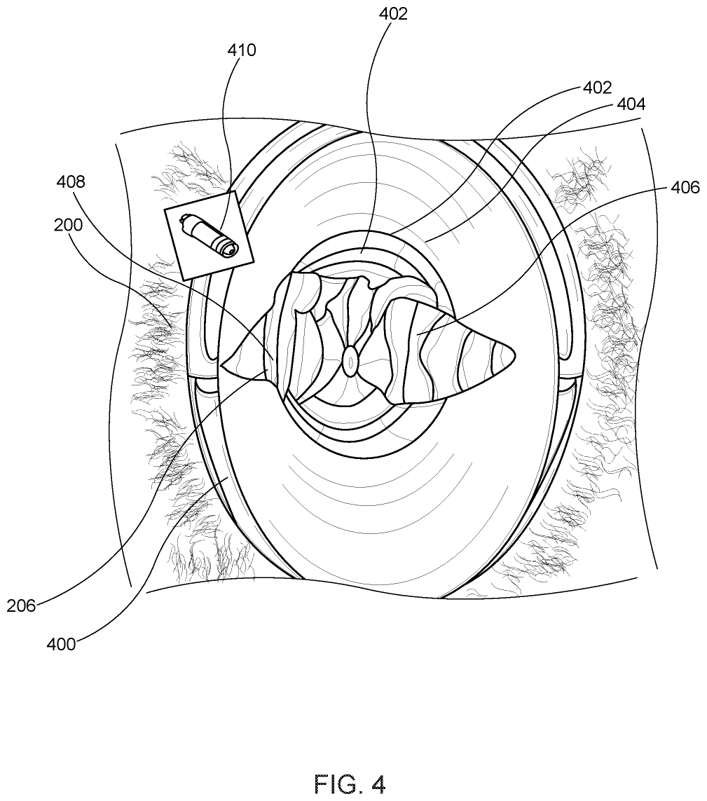

The method 100 may further include injecting, through the nozzles of the needle-free injector, the stem cell specimen and the platelet-rich plasma preparation directly on or near the vagina from multiple positions, multiple depths, and multiple angles, so that multiple injections are performed. In one non-limiting embodiment, the method more specifically involves a Step 116 of injecting, through at least one of the nozzles 410 of the needle-free injector, the stem cell specimen and the platelet-rich plasma (or normal saline) preparation into genital tissue at a 90° angle against tissue, along the urethral plane. As illustrated in FIG. 4, one of the nozzles is pressed firmly against one corner of the tented tissue of the vagina opening while the stem cell specimen is applied.

Additional injections are provided into: the anterior vaginal wall along the urethra plane 404; directly over the clitoris 610; and on both medial edge of the labia minora 616.

As FIG. 4 shows, the nozzle 410 is pointed along the urethral plane 404. The angle of the nozzle is at exactly 90° angle 402 against the tissue of the vaginal canal 204. It is necessary to reload new pre-filled nozzle into the needle-free injector, and position the nozzle directly over clitoris while pressing firmly, and injecting content. Specifically, the content is injected by pressing trigger on top of the needle-free injector. Then the nozzle is held steady for three seconds after the entire content has been injected into the vaginal tissue.

After this initial injection in the vaginal opening and clitoris, subsequent injections include injecting, through at least one of the nozzles, the stem cell specimen and the platelet-rich plasma (or normal saline) preparation laterally into the labia minora.

Thus, the steps for administrating the contents of the nozzle into the vagina include angling the nozzle laterally at a 90° angle 402 against labia minora approximately 1 cm laterally to the urethra opening. The steps further include reloading the pre-filled nozzle into the needle-free injector; and injecting laterally at 1 cm away from urethra opening on the other side. The method 100 may then also require firmly pressing the nozzle against labia minora at an angle as close to 90° as possible. The entire contents of the nozzle is then injected.

After complete deposition of stem cell specimen, the patient uses the shockwave device at home twenty-four hours after treatment, and three times per week thereafter for six weeks.

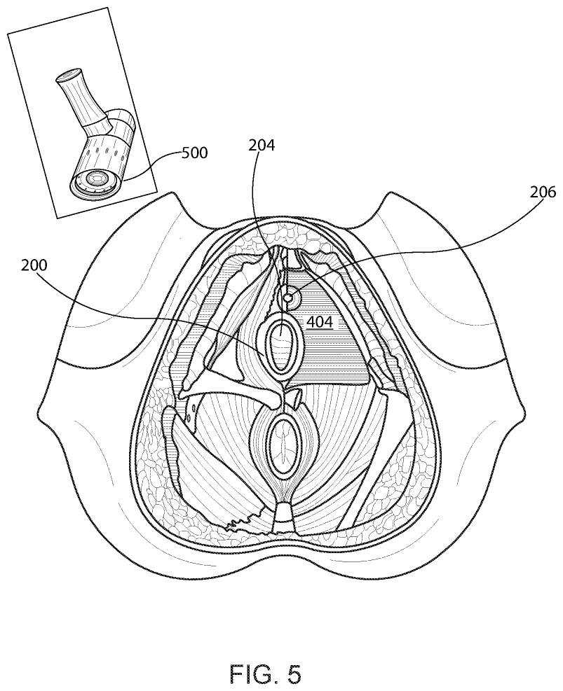

After complete deposition of stem cell specimen on the vagina, the patient uses the shockwave device at home twenty-four hours after treatment, and three times per week thereafter for six weeks. As shown in FIG. 5, a Step 118 includes reapplying, after a duration, the shockwave therapy to the vagina. In one embodiment, the patient uses the shockwave device 500 on the vagina 200 while at home, about twenty-four hours after treatment, and three times per week thereafter for six weeks.

For example, a shockwave home session instructions include: applying ultrasound gel over entire outer vaginal area; placing the head of shockwave device over entire areas for a total of 15 minutes; applying medium intensity with intermittent high intensity as tolerated; all while using a very slow circular motion with the shockwave device. (See FIG. 5) As in Step 104, the generated shockwave passes through the tissue and stimulates the cells dealing with connective tissue and bone healing.

A final Step 120 includes monitoring the patient for a post-procedure duration after administration of the stem cell specimen and the platelet-rich plasma preparation. The date of a maintenance session after administration of the stem cell specimen and platelet-rich plasma preparation depends on how long the sexual enhancement is sustained. This depends on the patient's age, health condition and lifestyle factors. However, intervals could range between 1-3 years. Each maintenance session follows the same procedure as one of above sessions.

Although the process-flow diagrams show a specific order of executing the process steps, the order of executing the steps may be changed relative to the order shown in certain embodiments. Also, two or more blocks shown in succession may be executed concurrently or with partial concurrence in some embodiments. Certain steps may also be omitted from the process-flow diagrams for the sake of brevity. In some embodiments, some or all the process steps shown in the process-flow diagrams can be combined into a single process.

It is recognized in the art that stem cell therapy is a complex and potentially dangerous procedure. Thus, the method may further include alternative Steps of providing emergency supplies in the event of untoward reactions. One such step is providing an Ammonium salt ampule for sniffing in case of vasovagal reaction. It may also be beneficial to provide Epinephrine 1:1000 (1 mg/ml), inject 0.5 mg IV or IM in case of anaphylactic reaction. The Epinephrine administration may repeat in 5 minutes if needed.

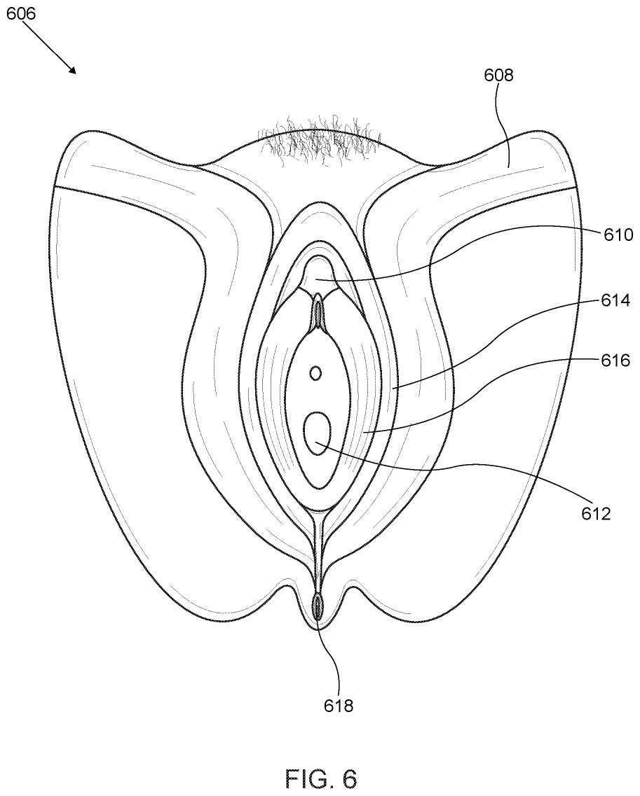

FIG. 6 illustrates an anatomical depiction of a full frontal view of the vaginal area 606 from the inner thigh 608 to the anus 618, upon which the Ammonium salt ampule and Epinephrine is applied, as discussed above. As shown in the illustration, the vaginal area 606 is located below the inner thigh 608, and includes a clitoris 610, below which the stem cell specimen is injected; an introitus 612 upon which the Stem cell specimen is injected through the vaginal canal; a labia majora 614 and a labia minora 616 that form loose flaps around the introitus 612.

Yet another emergency supply may include Benadryl 50 mg IV, which can be pushed over 2 minutes for anaphylactic reactions (may give after epinephrine dose). Finally, an Albuterol inhaler is provided in case the patient demonstrates shortness of breath during or after the method is applied. However, the method may also utilize other emergency systems and processes known in the art.

In one experimental application of the method, Allogeneic Mesenchymal Stem Cells Ameliorate Aging Frailty is addressed. The experiment is documented in A Phase II Randomized, Double-Blind, Placebo-Controlled ClinicalTrial—Tompkins et al, The Gerontological Society of America 2017. The experiment involved thirty patients average age of 75 with frailty received 100 vs 200 million IV allogeneic hMSCs vs Placebo. No therapy-related serious adverse events were recorded.

One advantage of the experiment is that the physical performance improved preferentially in the 100M group. The 6-minute walk test, short physical performance exam, and forced expiratory volume in 1 second improved in the 100M, not in the 200M or placebo groups. The female sexual quality of life improved in the 100M group. Further, immunologic improvement occurred in both the 100M and 200M groups. Serum TNF-α levels decreased in the 100M-group, B cell intracellular TNF-α improved in both the 100M and 200M groups. Finally, early and late activated T-cells were also reduced by MSC therapy.

These and other advantages of the invention will be further understood and appreciated by those skilled in the art by reference to the following written specification, claims and appended drawings.

Because many modifications, variations, and changes in detail can be made to the described preferred embodiments of the invention, it is intended that all matters in the foregoing description and shown in the accompanying drawings be interpreted as illustrative and not in a limiting sense. Thus, the scope of the invention should be determined by the appended claims and their legal equivalence.

What is claimed is:

1. A method for enhancing female sexual wellness with shockwave therapy and stem cells, the method comprising:
obtaining at least one metric of a female patient;
applying shockwave therapy to the vagina of the female patient;
developing a platelet-rich plasma preparation from the female patient;
obtaining a stem cell specimen;
introducing the stem cell specimen into a vial, the vial having a detachably attached needle-free injector defined by multiple nozzles;
introducing the platelet-rich plasma preparation into the vial;
drawing about 0.5 cubic centimeters of the stem cell specimen and the platelet-rich plasma preparation from the vial into each nozzle of the needle-free injector;
injecting, through at least one of the nozzles of the needle-free injector, the stem cell specimen and the platelet-rich plasma preparation directly on or near the vagina from multiple positions, multiple depths, and multiple angles, whereby multiple injections are performed;
reapplying, after a duration, the shockwave therapy to the vagina; and
monitoring the female patient for a post-procedure duration after administration of the stem cell specimen and the platelet-rich plasma preparation.

2. The method of claim 1, further comprising a step of obtaining consent from the female patient to proceed with the method.

3. The method of claim 1, further comprising a step of activating the platelet-rich plasma by positioning the platelet-rich plasma in a 37 degree Fahrenheit water bath for 1 to 3 minutes.

4. The method of claim 1, further comprising a step of applying an ultrasound gel over the vagina.

5. The method of claim 4, further comprising a step of applying a numbing cream over the vagina.

6. The method of claim 1, further comprising a step of heating a stem cell specimen vial containing the stem cell specimen to induce thawing by emerging the stem cell specimen vial in a 37° water bath, or by rolling the stem cell specimen vial against the palms of the hands.

7. The method of claim 1, wherein the step of introducing the stem cell specimen into a vial, further comprises drawing 2 cubic centimeters of the stem cell specimen into a 5 cubic centimeter syringe, the syringe being detachably attached to a 18 G 1½″ needle.

8. The method of claim 7, wherein the step of introducing the platelet-rich plasma preparation into the vial, further comprises drawing 1 cubic centimeter of the platelet-rich plasma preparation into the 5 cubic centimeter syringe, whereby the stem cell specimen and the platelet-rich plasma preparation are mixed.

9. The method of claim 8, further comprising a step of puncturing a rubber top on the vial with the needle-free injector.

10. The method of claim 9, wherein the needle-free injector comprises six nozzles.

11. The method of claim 1, further comprising a step of cleaning the vulval area of the vagina with a povodone-iodine swabstick.

12. The method of claim 1, further comprising a step of spreading open the vaginal canal of the vagina with a speculum.

13. The method of claim 1, wherein the step of injecting, through the needle-free injector, the stem cell specimen and the platelet-rich plasma preparation directly on or near the vagina from multiple positions, multiple depths, and multiple angles, further comprises: injecting, through at least one of the nozzles of the needle-free injector, the stem cell specimen and the platelet-rich plasma preparation at the open vaginal canal at a 90 degree angle relative to the urethral plane of the vagina.

14. The method of claim 13, wherein the step of injecting, through the needle-free injector, the stem cell specimen and the platelet-rich plasma preparation directly on or near the vagina from multiple positions, multiple depths, and multiple angles, further comprises: injecting, through at least one of the nozzles of the needle-free injector, the stem cell specimen and the platelet-rich plasma preparation at a 9 o'clock position, and at a 3 o'clock position relative to the vaginal wall of the vagina, and at a depth of 1 to 2 inches into the vaginal canal.

15. A method for enhancing female sexual wellness with shockwave therapy and stem cells, the method comprising:
obtaining at least one metric of a female patient;
applying shockwave therapy to the vagina of the female patient;
developing a platelet-rich plasma preparation from the female patient;
obtaining a stem cell specimen;
introducing the stem cell specimen into a vial, the vial having a detachably attached needle-free injector defined by multiple nozzles;
introducing the platelet-rich plasma preparation into the vial;
drawing about 0.5 cubic centimeters of the stem cell specimen and the platelet-rich plasma preparation from the vial into each nozzle of the needle-free injector;
injecting, through at least one of the nozzles of the needle-free injector, the stem cell specimen and the platelet-rich plasma preparation at the open vaginal canal at a 90 degree angle relative to the urethral plane of the vagina;
injecting, through at least one of the nozzles of the needle-free injector, the stem cell specimen and the platelet-rich plasma preparation at a 9 o'clock position, and at a 3 o'clock position relative to the vaginal wall of the vagina, and at a depth of 1 to 2 inches into the vaginal canal;
reapplying, after a duration, the shockwave therapy to the vagina; and
monitoring the female patient for a post-procedure duration after administration of the stem cell specimen and the platelet-rich plasma preparation

16. The method of claim 15, further comprising a step of applying an ultrasound gel over the vagina.

17. The method of claim 16, further comprising a step of applying a numbing cream over the vagina.

18. The method of claim 17, further comprising a step of heating a stem cell specimen vial containing the stem cell specimen to induce thawing by emerging the stem cell specimen vial in a 37° water bath, or by rolling the stem cell specimen vial against the palms of the hands.

19. The method of claim 18, wherein the step of introducing the stem cell specimen into a vial, further comprises drawing 2 cubic centimeters of the stem cell specimen into a 5 cubic centimeter syringe, the syringe being detachably attached to a 18 G 1½″ needle.

20. A method for enhancing female sexual wellness with shockwave therapy and stem cells, the method consisting of:
obtaining at least one metric of a female patient;
applying shockwave therapy to the vagina of the female patient;
developing a platelet-rich plasma preparation from the female patient;
obtaining a stem cell specimen;
introducing about 2 cubic centimeters of the stem cell specimen into a vial having a detachably attached needle-free injector, the needle-free infector comprising six nozzles;
introducing about 1 cubic centimeter the platelet-rich plasma preparation into the vial;
drawing about 0.5 cubic centimeters of the stem cell specimen and the platelet-rich plasma preparation into each nozzle of the needle-free injector;
cleaning the vulval area of the vagina with a povodone-iodine swabstick;
spreading open the vaginal canal of the vagina with a speculum;
injecting, through at least one of the nozzles of the needle-free injector, the stem cell specimen and the platelet-rich plasma preparation at the open vaginal canal at a 90 degree angle relative to the urethral plane of the vagina;
injecting, through the needle-free injector, the stem cell specimen and the platelet-rich plasma preparation at the open vaginal canal at a 9 o'clock position, and at a 3 o'clock position relative to the vaginal wall of the vagina, and at a depth of 1 to 2 inches into the vaginal canal;
reapplying, after a duration, the shockwave therapy to the vagina; and
monitoring the patient for a post-procedure duration after administration of the stem cell specimen and the platelet-rich plasma preparation.
